(19) United States
(12) Patent Application Publication
(10) Pub. No.: US 2014/0315230 A1
(43) Pub. Date: Oct. 23, 2014

(54) PROCESS FOR RAPIDLY SCREENING ENZYME AND ENZYME MIXTURES FOR THE HYDROLYSIS OF LIGNO-CELLULOSIC BIOMASS

(71) Applicant: C5-6 Italy S.R.L., Tortona (IT)

(72) Inventors: Laura Volpati, Tortona (IT); Stefano Paravisi, Tortona (IT); Beatriz Rivas Torres, Arenzano (IT)

(21) Appl. No.: 14/359,349

(22) PCT Filed: Nov. 23, 2012
(86) PCT No.: PCT/IB2012/056677

§ 371 Date: May 20, 2014

(30) Foreign Application Priority Data
Nov. 23, 2011 (IT) TO2011A001076

(51) Int. Cl. C12Q 1/34

(52) U.S. Cl. CPC C12Q 1/34
USPC 435/18

(57) ABSTRACT

The invention is to a novel method for evaluating the hydrolysis conversion of a enzyme or enzyme mixture relative to a specific ligno-cellulosic biomass. The method evaluates the hydrolysis conversion of a enzyme or enzyme mixture relative to a specific ligno-cellulosic biomass by preparing a pretreated ligno-cellulosic biomass suspension, a buffer solution and an enzyme or enzyme mixture solution; dispensing a portion of one or more of the three components into a plurality of test vials, subjecting one or more of the plurality of test vials to varying incubation periods, reducing the rate of incubation at the end of each incubation period, and analyzing the resulting samples for dissolved sugars to determine the rate of reaction.

### BACKGROUND

The current test for screening enzymes and enzyme mixtures for the hydrolysis of ligno-cellulosic biomass is the FPU test in small scale. However, the FPU test is limited to a cellulose simulant and provides an overall limited suggestion of the applicability of the enzymatic mixture to a ligno-cellulosic material. One current test for an actual ligno-cellulosic material is a large scale (3.5 L) stirred reactor test requiring significant amounts of enzyme and time. This current test is not useful for conducting for screening of a large set of enzymes and enzymes mixtures in an economic way, as required by industrial application. There exists therefore, the need for a process to evaluate enzymes and enzyme mixtures in a more rapid and efficient manner, reducing the time and the amount of materials used for testing.

### SUMMARY

This specification discloses a process for evaluating the hydrolysis conversion of a enzyme or enzyme mixture relative to a specific ligno-cellulosic biomass. The method disclosed in this specification includes the steps of; a) preparing a pretreated ligno-cellulosic biomass suspension, a buffer solution and an enzyme or enzyme mixture solution, b) dispensing a portion of the pre-treated ligno-cellulosic suspension and buffer solution into at least two test vials of a plurality of test vials, c) adding a portion of the enzyme or enzyme mixture solution to at least one test vial containing the pre-treated ligno-cellulosic biomass while leaving at least one test vial void of enzyme or enzyme mixture solution, d) dispensing the buffer solution into at least one test vial which is void and will remain void of the enzyme or enzyme mixture solution and the ligno-cellulosic biomass suspension, e) analyzing the at least one test vial which is void of enzyme or enzyme mixture solution but contains pre-treated ligno-cellulosic biomass and buffer for soluble sugars prior to incubating the test vials, f) incubating the test vials for a first time at a first temperature and analyzing at least one of the test vials containing the enzyme or enzyme mixture solution for soluble sugars at the first time, g) incubating the remaining test vials for a second time and analyzing at least one of the remaining test vials containing the enzyme or enzyme mixture solution for soluble sugars, h) and incubating the remaining test vials and analyzing at least one test vial at each of several pre-determined times of incubation for dissolved sugars to determine the rate of reaction.

It is also disclosed that the volume of the portion of the biomass suspension may less than 2.0 ml, preferably less than 1.0 ml, more preferably less than 0.5 ml, even more preferably less than 0.3 ml, yet even more preferably less than 0.2 ml, being less than 0.1 ml the most preferred value.

It is further disclosed that the dry matter percent content by weight of the ligno-cellulosic suspension in the test vials may be higher than a value selected from the group consisting of 1, 2, 4, 10, 12, 15, 17, and 20.

It is also disclosed that the test vials may be analyzed for soluble sugars by means of High-Performance Liquid Chromatography.

It is further disclosed that wherein the test vials may be analyzed for soluble sugars by means of optical absorbance and that a reagent may be added to each vial of the plurality of test vials before analyzing the test vials. The reagent may also quench the incubation of the test vials containing the enzyme or enzyme mixture.

It is also disclosed that the reagent may comprise dinitrosalicylic acid.

It is further disclosed that the optical absorbance of the liquid fraction of the at least one test vial may be analyzed at a unique wavelength.

### DETAILED DESCRIPTION

Described in the specification is a method for evaluating the hydrolysis conversion of a enzyme or enzyme mixture relative to a specific ligno-cellulosic biomass. The method as described is useful for the rapid and efficient evaluation of enzymes and enzyme mixtures for the hydrolysis conversion of a composition relative to a specific ligno-cellulosic biomass. The disclosed process requires a very small amount of solutions, comprising a buffer solution and an enzymes or enzyme mixture solution, and may be conducted in an automatic or semiautomatic way, thereby enabling a method for testing a large amount of enzymes or enzyme mixture in a short time and at a low cost.

The process follows the steps of; a) preparing a pretreated ligno-cellulosic biomass suspension, a buffer solution and an enzyme or enzyme mixture solution, b) dispensing a portion of the pre-treated ligno-cellulosic suspension and buffer solution into at least two test vials of a plurality of test vials, c) adding a portion of the enzyme or enzyme mixture solution to at least one test vial containing the pre-treated ligno-cellulosic biomass while leaving at least one test vial void of enzyme or enzyme mixture solution, d) dispensing the buffer solution into at least one test vial which is void and will remain void of the enzyme or enzyme mixture solution and the ligno-cellulosic biomass suspension, e) analyzing the at least one test vial which is void of enzyme or enzyme mixture solution but contains pretreated ligno-cellulosic biomass and buffer for soluble sugars prior to incubating the test vials, f) incubating the test vials for a first time at a first temperature and analyzing at least one of the test vials containing the enzyme or enzyme mixture solution for soluble sugars at the first time, g) incubating the remaining test vials for a second time and analyzing at least one of the remaining test vials containing the enzyme or enzyme mixture solution for soluble sugars, h) and incubating the remaining test vials and analyzing at least one test vial at each of several pre-determined times of incubation for dissolved sugars to determine the rate of reaction.

The test vials are characterized as any vial which is capable of containing a liquid such as a test tube, an Eppendorf tube or a flask.

Preparing the pretreated ligno-cellulosic biomass suspension can follow the steps as outlined in WO-2010/113129.

Preparing a reagent may optionally be achieved by preparing a 2N NaN₃ solution. Preparing a 2N NaN₃ solution may be achieved by dissolving 13.002 g of NaN₃ in ultrapure water up to a final volume of 100 mL.

Preparing a reagent may also be achieved by preparing a 1M CaCl₂ solution. Preparing a 1M CaCl₂ solution may be achieved by dissolving 9.389 g of CaCl₂ in ultrapure water up to a final volume of 100 mL.

Preparing a reagent may also be achieved by preparing a 1M Dithiothreitol (DTT) solution. Preparing a 1M DTT solution may be achieved by dissolving 69.41 g of dithiothreitol in distilled water up to a final volume of 10 mL, preparing 0.5 mL of aliquots and storing at −20° C.

Preparing the buffer solution may be achieved by preparing a citrate buffer solution. A citrate buffer solution may be prepared as a 1N stock solution by mixing preferably 210 g of citric acid monohydrate with 750 ml of ultrapure water and adding between 50 and 60 g of NaOH, the amount of NaOH determined by the amount necessary to reach a pH of 4.5. A 0.05 M working solution is then prepared by mixing 50 ml of the 1N stock solution with 950 ml of ultrapure water and adding 1M CaCl₂ (1 ml) and/or 0.45M DTT (22 ml), depending upon the enzyme characteristics, and adjusting the pH to the required value.

Contained in each of the plurality of the test vials is a ligno-cellulosic suspension which may be in the form of a substrate solution. Inventors have found that, being the use of a small amount of ligno-cellulosic suspension desirable for the rapid screening of a large set of enzymes and enzymes mixtures, the preparation and handling of such a small amount of ligno-cellulosic suspension may conduct to misleading results. The use of the ligno-cellulosic suspension in a small volume requires that the ligno-cellulosic suspension in each test vial is statistically representative of a larger sample. In particular, it is required that the concentration of the biomass in the biomass suspension used in each vial is substantially the same concentration of that one in a reference biomass suspension volume of 1 liter. In this way, it is preserved the condition that the amount of enzymes or enzyme mixture per gram of substrate in each test vials is not affected by the small volume of biomass suspension in the test vials. Thereby the preparation of the biomass suspension is a critical step not disclosed in the prior art. The pre-treated ligno-cellulosic biomass may be subjected to a size reduction treatment. Preferably, the size reduction treatment comprises mechanical treatment which impresses cutting forces and/or shear forces to the pre-treated ligno-cellulosic biomass. The volume of the pre-treated ligno-cellulosic suspension in the test vials may be very small with respect to methods disclosed in the prior art, being less than 2.0 ml, preferably less than 1.0 ml, more preferably less than 0.5 ml, even more preferably less than 0.3 ml, yet even more preferably less than 0.2 ml, being less than 0.1 ml the most preferred value. The dry matter content by weight of the pre-treated ligno-cellulosic biomass suspension in the test vials may be higher than 1, more preferably higher than 2, even more preferably higher than 4, even yet more preferably higher than 10, even yet more preferably higher than 12, even yet more preferably higher than 15, most preferably higher than 17, being higher than 20 the even most preferred value. Preparing a substrate solution may be done by mixing 2.200 g of pretreated ligno-cellulosic material with 17.90 ml of ultrapure water, 70 μL it of 2M NaN₃ solution and 1 mL of 1N citrate buffer having a pH of 4.5. Depending on the enzyme characteristics preparing the substrate solution may further require adding 1M CaCl₂ (200 μL) and/or 0.45M DTT (444 μL). Preparing the substrate solution further requires keeping the material suspension in agitation using a magnetic stirrer. Preparing the substrate solution may further require adjusting the pH to the required value.

Preparing the enzyme or enzyme mixture solution is done according to common preparation techniques depending on enzyme or enzyme mixture solution. Common preparation techniques are known to people skilled in the art.

The steps of adding a portion of the pre-treated ligno-cellulosic suspension and/or buffer solution may require adding the components to a 1.5 mL test vial. The pre-treated ligno-cellulosic suspension may be added in the form of a substrate solution. The substrate solution may be added in amounts of up to 0.3 ml. The buffer solution may be added in amounts varying between 0.2 ml and 0.35 ml.

Where the purpose of the process is to study the kinetic of the reaction of the enzyme or enzyme mixture solution it is preferred to prepare six test vials for each enzyme or enzyme mixture solution tested. Where the purpose of the process is to quantify the final yield of the enzymatic reaction after a fixed time it is preferred to prepare three test vials for each enzyme or enzyme mixture solution tested. In either case, it is necessary to prepare two additional test vials containing ligno-cellulosic suspension and/or buffer solution, but no enzyme or enzyme mixture solution. Prior to beginning any incubation period, the first additional test vial may be analyzed according to the procedures outlined below for characterizing the selected sample. Following an incubation period for an incubation time which is at least as long as the longest incubation time of a test vial containing an enzyme or enzyme or enzyme mixture solution, the second additional test vial may be analyzed according to the procedures outlined below for characterizing the selected sample.

After preparing each of the plurality of test vials with ligno-cellulosic suspension and/or buffer solution, the following procedures are necessary. Preferably, the plurality of test tubes are placed in a thermomixer at a temperature selected to match enzyme optimum. The thermomixer is preferably set to 900 rpm. Preferably the temperature of the test vials are equilibrated in the thermomixer for at least five minutes prior to adding the enzyme or enzyme mixture solution. The enzyme or enzyme mixture solution may then be added in amounts of up to 0.15 ml. After adding the enzyme or enzyme mixture solution it is necessary to incubate the test vials for an incubation period in the thermomixer at a set temperature and at a set rpm. Preferably the set temperature during the incubation period is at least 50° C. Preferably the set rpm during the incubation period is at least 900 rpm.

The incubation period may be stopped at a different incubation time for each test vial, and the samples analyzed to quantify sugar released. By way of example, but not limitation, the incubation time for the first test vial may be at least one hour. By way of example, but not limitation, the incubation time for the second test vial may be at least six hours. By way of example, but not limitation, the incubation time for the third test vial may be at least twenty-two hours. By way of example, but not limitation, the incubation time for the fourth test vial may be at least twenty-nine hours. By way of example, but not limitation, the incubation time for the fifth test vial may be at least forty-six hours. By way of example, but not limitation, the incubation time for the sixth test vial may be at least sixty-six hours.

At the end of the incubation time, the soluble sugars content of the test vials may be analyzed by means of High-Performance Liquid Chromatography (HPLC). The selected test vial is removed from the thermomixer, and incubated at 99° C. for a time of seven minutes at 900 rpm. The selected test vial is then transferred to a cold ice-water bath to cool for a time of at least ten minutes. Following the ice-water bath, the selected sample is then centrifuged at 4° C. at 12000 rpm for at least four minutes. After centrifuging, the liquid fraction of the selected sample is moved to an HPLC test tube. In some cases a dilution may be helpful for the analysis.

Once the liquid fraction of the selected sample is moved to the HPLC tube, it can be characterized for sugar content and composition (e.g. monomers and oligosaccharides) using an HPLC. The process of characterizing the liquid fraction of the selected sample using HPLC may be performed after post-hydrolysis at 4% H₂SO₄ at 121° C. for one hour. Preferably, the process of characterizing the liquid fraction of the selected sample will occur both before and after performing post- hydrolysis at 4% H₂SO₄ at 121° C. for one hour.

At the end of the incubation time, the soluble sugars content of the test vials may be analyzed by means of optical absorbance. Preferably a reagent is added to the test vials containing the enzyme or enzyme mixture to quench the reaction rate. Preferably, the reagent is a solution comprising 3,5-Dinitrosalicylic acid. Optical absorbance is read at preferably 540 nm.

Quenching the reaction is characterized by sufficiently reducing the rate of reaction so that the analysis of the composition of the vials can be accurately obtained corresponding to the time the vials are removed from the temperature controlled environment. Typically, the reaction rate will be reduced to a rate in the range of 0 to 20% of the rate of the reaction being evaluated. More preferably, the rate will be in the range of 0 to 10%, with 0 to 5% being even more preferred, with 0 to 2% being the most preferred.

One method of preparing a reagent may involve the steps of; a) dissolving an amount of distilled water with an amount of dinitrosalicylic acid and an amount of sodium hydroxide, b) adding an amount of Rochelle salts, c) adding an amount of phenol and, d) adding an amount of sodium metabisulfite (Na₂S₂O₅). Preferably the amount of distilled water is 1416 ml. Preferably the amount of dinitrosalicylic acid is 10.6 g. Preferably the amount of sodium hydroxide is 19.8 g. Rochelle salts may include, but are not limited to sodium potassium tartrate and KNaC₄H₄O₆.4H₂O. Rochelle salts are preferably added in an amount of 306 g. Phenols are preferably added in an amount of 7.6 ml. Sodium metabisulfite (Na₂S₂O₅) is preferably added in an amount of 8.3 g.

Another method of preparing a reagent may involve preparing an Na₂CO₃ solution by dissolving an amount of Na₂CO₃ in ultrapure water to a final volume. Preferably the amount of Na₂CO₃ is 10.6 g. Preferably the final volume is 100 ml.

After quenching the reaction the sample may be cooled in ice for a time of preferably 10 minutes.

If dilution is needed, the step of analyzing the optical absorbance of the liquid fraction of at least one test vial of the plurality requires placing preferably a portion of the sample directly in a well of preferably a 96-well plate and adding ultrapure water and mixing the sample vigorously to obtain a properly diluted sample.

We claim:

1-9. (canceled)

10. A process for evaluating the hydrolysis conversion of a enzyme or enzyme mixture relative to a specific ligno-cellulosic biomass, the process comprising the steps of:
a. Preparing a pre-treated ligno-cellulosic biomass suspension, a buffer solution and an enzyme or enzyme mixture solution,
b. Dispensing a portion of the pre-treated ligno-cellulosic suspension and buffer solution into at least two test vials of a plurality of test vials,
c. Adding a portion of the enzyme or enzyme mixture solution to at least one test vial containing the pre-treated ligno-cellulosic biomass suspension, leaving at least one test vial void of enzyme or enzyme mixture solution,
d. Dispensing the buffer solution into at least one test vial which is void and will remain void of the enzyme or enzyme mixture solution and the pretreated ligno-cellulosic biomass suspension,
e. Analyzing the at least one test vial which is void of enzyme or enzyme mixture solution but contains pre-treated ligno-cellulosic biomass suspension and buffer solution for soluble sugars prior to incubating the test vials,
f. incubating the test vials for a first time at a first temperature and analyzing at least one of the test vials containing the enzyme or enzyme mixture solution for soluble sugars at the first time,
g. incubating the remaining test vials for a second time and analyzing at least one of the remaining test vials containing the enzyme or enzyme mixture solution for soluble sugars,
h. incubating the remaining test vials and analyzing at least one test vial at each of the several pre-determined times of incubation for dissolved sugars to determine the rate of reaction.

11. The process of claim 10, wherein the volume of the portion of the biomass suspension in each test vials is less than a value selected from the group consisting of 2.0, 1.0, 0.5, 0.3, 0.2, and 0.1 mL.

12. The process of claim 10, wherein the dry matter percent content by weight of the ligno-cellulosic suspension in the test vials is higher than a value selected from the group consisting of 1, 2, 4, 10, 12, 15, 17, and 20.

13. The process of claim 11, wherein the dry matter percent content by weight of the ligno-cellulosic suspension in the test vials is higher than a value selected from the group consisting of 1, 2, 4, 10, 12, 15, 17, and 20.

14. The process of claim 10, wherein the at least one of the test vials of steps e, f, g, and h are analyzed by means of High-Performance Liquid Chromatography.

15. The process of claim 10, wherein the at least one of the test vials of steps e, f, g, and h are analyzed by means of optical absorbance.

16. The process of claim 15, wherein in step e, f, g, and h a reagent is added to each vial of the plurality of test vials before analyzing the test vials.

17. The process of claim 16, wherein the reagent quenches the incubation of the test vials containing the enzyme or enzyme mixture.

18. The process of claim 17, wherein the reagent comprises dinitrosalicylic acid.

19. The process of claim 15, wherein the optical absorbance of the liquid fraction of the at least one test vial is analyzed at a unique wavelength.

20. The process of claim 16, wherein the optical absorbance of the liquid fraction of the at least one test vial is analyzed at a unique wavelength.

21. The process of claim 17, wherein the optical absorbance of the liquid fraction of the at least one test vial is analyzed at a unique wavelength.

22. The process of claim 18, wherein the optical absorbance of the liquid fraction of the at least one test vial is analyzed at a unique wavelength.
